(19) United States
(12) Patent Application Publication
(10) Pub. No.: US 2012/0316572 A1
(43) Pub. Date: Dec. 13, 2012

(54) TRANSCERVICAL EXCISION AND REMOVAL OF TISSUE

(75) Inventors: Peter L. Rosenblatt, West Newton, MA (US); Anthony J. DiSciullo, Westwood, MA (US)

(73) Assignee: NEW ENGLAND ASSOC OF GYNECOLOGIC LAPAROSCOPI

(21) Appl. No.: 13/446,757

(22) Filed: Apr. 13, 2012

Related U.S. Application Data
(63) Continuation of application No. 12/745,645, filed on Jun. 1, 2010, now abandoned.
(60) Provisional application No. 61/004,908, filed on Nov. 30, 2007.
(60) Provisional application No. 61/087,786, filed on Aug. 11, 2008.

(51) Int. Cl. A61B 17/42

(52) U.S. Cl. 606/119

(57) ABSTRACT

A hysterectomy method may include: severing a patient's uterus from the patient's cervix; then coring the cervix; and then morcellating the severed uterus using a morcellator inserted through the cored cervix.

### CROSS-REFERENCE TO RELATED APPLICATIONS

This application claims the benefit of U.S. application Ser. No. 12/745,645, filed Jun. 1, 2010, which the U.S. national stage entry of PCT/US08/085164, filed Dec. 1, 2008, claims the benefit of U.S. Provisional Application Nos. 61/004,908, filed Nov. 30, 2007, and 61/087,786, filed Aug. 11, 2008, both of which are hereby incorporated herein by reference.

### FIELD

The disclosed systems and methods relate generally to systems and methods for performing transcervical or transvaginal morcellation of tissue.

### BACKGROUND

Surgery to remove masses in the abdomen, such as a uterus or myoma, may be performed in a minimally-invasive manner, which usually involves morcellating the tissue and removing it through an enlarged port site in the abdomen. For example, laparoscopic supracervical hysterectomy involves mobilization of the bladder off the uterus, control of vascular pedicles (specifically the uterine and ovarian arteries), transection of the uterus from the cervix, and then removal of the uterus from the abdominal cavity. This last step is most often performed with the use of an electromechanical morcellator. Strips of uterine tissue are drawn through the morcellation device until the entire specimen has been removed. Although this technique obviates the need for laparotomy incision, it does require a larger port site incision than would be needed if the morcellator were not used through the abdomen. Larger port sites create more pain, due to the increased size of the incision, and from additional sutures that are commonly used to reapproximate the abdominal wall fascia to prevent bowel herniation. In addition, one of the arguments against supracervical hysterectomy (compared to complete hysterectomy) has been the persistent potential for the development of cervical dysplasia and carcinoma. By leaving the transitional zone on the cervix, as well as the endocervix, laparoscopic supracervical hysterectomy requires that the patient continues with routine PAP smears or other tests of cervical cytology screening. Another criticism of supracervical hysterectomy is the potential for persistent cyclic bleeding, which may result from hormonally active cells being left in the endocervical canal, even despite some surgeons' efforts to cauterize or remove the upper aspect of the endocervix after amputation of the uterus from the cervix. One procedure, classic intrafascial supracervical hysterectomy (CISH), involves coring out the endocervix and endometrial cavity and then performing a supracervical hysterectomy, but morcellation of the uterine fundus is performed through a large port in the abdominal wall.

### SUMMARY

The present disclosure provides systems and methods for performing morcellation of masses through a vaginal approach. In one embodiment, the procedure involves coring out the endocervix, thereby also removing part of the transformation zone on the cervix, with the use of a morcellator or handheld coring tube, and then using a morcellator placed through the cervix to complete the morcellation of the specimen, such as the uterine fundus.

### BRIEF DESCRIPTION OF THE DRAWINGS

The drawings illustrate principles of the systems and methods disclosed herein and are not necessarily to scale. Implied absolute or relative dimensions are not limiting but are instead provided for illustrative purposes.

FIGS. 1A-B demonstrate one design of the instrument, with a central rod for locating the endocervical canal. In this embodiment, a flat screw is located in a fixed position on the rod and is screwed into the cervical tissue, in order to be able to apply traction on the cervix during coring out of the endocervical canal with the morcellator device.

FIG. 2 illustrates the instrument described above with a flat screw that is movable on the rod but can be locked into position on the rod.

FIG. 3 demonstrates another embodiment of the cervical rod in which there are flexible or stiff barbs located along the outside of the length of the rod

FIGS. 4A-1 and 4A-2 illustrate curved hooks located at the distal end of the rod, which are deployed in order to grasp the cervical core tissue. FIG. 4B demonstrates a rod with a stabilization ring (8) along the shaft.

FIG. 5 demonstrates an instrument that has both a central rod and a grasping device to hold on to the cervical tissue.

FIGS. 6A-B illustrate a rod that has a mechanism that opens up prongs at the distal end.

FIG. 7 illustrates a balloon that may be inflated after passage through the cervix.

FIG. 8 demonstrates the placement of a video monitor at the head of the patient's bed with a reversed image, in order to facilitate morcellation for the surgeon operating the morcellator.

FIG. 9 demonstrates the morcellation of the specimen, using a grasping instrument placed through the morcellator to hold on to the specimen, and assisted by laparoscopic instruments.

FIG. 10 demonstrates a needle with jaws that can be opened, placed through the cervix and up into the abdominal cavity.

FIGS. 11 and 11B demonstrate a needle with a spring loaded hook, placed through the cervix and up into the abdominal cavity.

FIG. 12 demonstrates a needle with a spring loaded notch, placed through the cervix and up into the abdominal cavity.

FIG. 13 illustrates the process of leaving a suture placed on one side of the cervix and up into the abdominal cavity, and then placing another needle through the contralateral side of the cervix and grasping the other end of the suture.

FIG. 14 illustrates a blunt device with a collar placed into the cervix with a needle that can be advanced through holes on either side of the instrument.

FIG. 15 shows a grasping needle placed through one side of the cervix and into the abdominal cavity, and a suture can be loaded onto this grasping needle.

FIG. 16 illustrates a hollow needle placed through the cervix with a snare loop opened up once inside the abdominal cavity, through which a suture can be loaded.

FIG. 17 illustrates a snare loop on one side and a hollow needle with a pre-loaded suture within the needle on the other side of the blunt probe.

FIG. 18 illustrates the electrocautery function of the blunt probe, in order to coagulate the inner portion of the cervical core, if needed for control of bleeding.

FIG. 19 illustrates a separate handheld coring device used to core out the cervical canal and surrounding tissue.

FIG. 20 illustrates a cervical coring device that is detachable from an electromechanical device.

FIG. 21 illustrates a cervical trocar device with a proximal valve.

FIG. 22 illustrates a cervical trocar device with a protective cover applied over the sharp blade after coring out the cervix.

FIG. 23 demonstrates a cervical trocar device with a protective inner sleeve inserted beyond the sharp blade.

FIG. 24 shows an obturator instrument in the cervix with a collar that is brought up against the cervix. The collar has two channels located on either side, through which a needle can be placed and will then traverse the cervical tissue. The distal tip of the needle will then come through the cervix and can be used to deliver or retrieve suture that is used to close the cervix.

FIG. 25 demonstrates a suturing device that automatically delivers a needle tip through the cervical tissue and catches the needle tip in the cage, located in the shaft of the instrument. This can be done on either side of the cervical defect. The suture is then tied down.

FIG. 26 demonstrates the attachment of the suturing device to the trocar, which exposes the needle path and the cage where the needle will be captured.

FIG. 27 illustrates a barrel-shaped plug that can be placed in the cervical defect.

FIG. 28 illustrates a barrel-shaped plug that has two wings which flare out after being placed through the cervix.

### DETAILED DESCRIPTION

For clarity and convenience, a number of exemplary embodiments will be described relating to a particular anatomic site, the female pelvis. However, it will be readily apparent to one of ordinary skill in the art that the disclosed systems and methods may be employed in a wide variety of anatomical settings to treat a broad range of abnormalities.

An exemplary method for cervical coring with supracervical hysterectomy may include:
Control of vascular supply of the upper pedicles (either the infundibulopelvic ligaments, if oophorectomy is performed with the hysterectomy, or the utero-ovarian ligaments, if the ovaries are preserved);
Control of the ascending uterine arteries;
Amputation of the uterine corpus from the cervix;
Vaginal transcervical sounding or grasping of the endocervical canal;
Coring out of the cervix and removal of this tissue;
Transcervical morcellation of the uterine corpus; and
Closure of the cervical defect.

In one exemplary embodiment, an instrument with a rod is inserted transvaginally through the morcellation device and through the cervix after amputation of the uterus. This rod is used to define the location and axis of the endocervical canal and can be visualized laparoscopically. Once the rod is placed through the cervix, a flat screw device mounted on the rod is applied to the cervix, thus permitting traction to be applied to the cervix. This screw may be fixed or movable on the rod. At this point, morcellation of the endocervix is performed with the morcellation device.

Alternatively, the rod could have flexible or stiff barbs or tines, made from materials such as plastic or metal, that permit placement through the cervix but engage on the tissue when traction is placed on the rod, thus facilitating removal of the cervical core after morcellation is performed. The rod placed through the cervix could also use other methods to engage the cervical tissue, such as suction or an adhesive material.

In another embodiment, curved hooks could be deployed at the distal end of the rod, or along the shaft of the rod, once the rod is placed through the cervix, which would engage the cervical tissue and facilitate extraction of the tissue following morcellation of the cervical core. Alternatively, the rod may be straight and not have any devices that grab onto the tissue. Regardless of the presence or type of grasping devices, the rod may also have a stabilization ring or rings that are similar in diameter to the inner diameter of the morcellator. These stabilization rings prevent the morcellator from taking a different path than the one created by the rod, which may compromise the accurate removal of the endocervical canal and adjacent tissue. Alternatively, the rod could have a lower section that is wider than the rod itself with a diameter that is slightly smaller than the inner diameter of the shaft of the morcellator (or coring blade), so that the rod does not deviate from side to side.

In another embodiment, an instrument that has both a central rod (for placement through the cervix) and a grasping device can be placed through the morcellator to both locate the cervix and hold onto the cervix in order to facilitate coring out of the endocervix.

In another embodiment, the central rod may have the ability to spread open at the distal end, with one of a number of devices, such as prongs that spring open, or an inflatable balloon, which help to stabilize the cervical tissue.

Once the endocervical core is removed, the morcellator may be left in place in order to morcellate the uterus. Morcellation is performed in standard fashion, with a grasping instrument, such as a tenaculum, inserted through the morcellator. This may be facilitated with the use of a video monitor with a reversed image located near the head of the surgical table. In this manner, an assistant standing between the patient's legs can see a “mirror image” of the abdomen and the morcellator that they are operating.

The morcellating device used may need to have an elongated shaft, since it must traverse the vaginal canal and the cervix in order to be used to morcellate the specimen, such as the uterine fundus after a laparoscopic supracervical hysterectomy.

Alternatively, a separate coring device can be used to core out the central portion of the cervix. This may be a handheld or electromechanical device that has a hollow tube with a sharp leading edge that may or may not be serrated. The coring device may be detachable from an electromechanical device, once the coring is complete. Once the coring is complete, the coring device may be removed and the morcellator is placed through the cervix to perform morcellation. The electromechanical morcellator may be the same device that places the separate coring device, and then morcellation of the uterus is performed with a different attachment connected to the electromechanical device.

In another embodiment, once the separate coring device completes the coring of the cervix, the coring device is left in place and is used as an access instrument, or trocar, through which the morcellator may be placed. This trocar may have one or more features, such as a proximal valve to prevent loss of abdominal pneumoperitoneum, similar to standard laparoscopic trocars. The trocar may also define one or more channels in its wall, inner face, or outer face; needles can be advanced through the channels for suturing closed the cored cervix. The valve mechanism may be built in to the instrument or may be attached to the proximal end of the instrument after the coring has been accomplished. The sharp end of the trocar may be left just inside the abdominal cavity (just beyond or just inside the cervix) or may be protected, by one of several methods. The sharp blade may have a mechanism that retracts the sharp blade, or conversely an inner tube may be placed within the outer sharp tube and extends just beyond the outer, sharp tube. This inner tube may screw, snap or otherwise fix into place so that it is stabile and does not move in relationship to the outer, sharp blade.

The morcellator may then be placed through the trocar and the specimen is removed in standard fashion.

Once the morcellation is completed, a balloon-tipped catheter may be placed through the cervix and inflated in order to maintain the pneumoperitoneum until the surgeon elects to close the endocervical canal defect. If the trocar is used, the trocar itself can be used to maintain the pneumoperitoneum until the surgeon closes the cervical defect.

If there is any bleeding noted from the cervix after the coring out process, radiofrequency energy may be used to coagulate the inner walls of the cervical core. This may be done by monopolar cautery located on the blade of the morcellator. Alternatively, the blunt probe (described below for the suture closure of the cervix) may incorporate monopolar or bipolar electrocautery electrode(s) located along the distal length of the instrument (distal to the collar) that may be used to coagulate the inner lining of the cervix. Alternatively, the cervical trocar may incorporate monopolar or bipolar electrocautery electrode(s) area near the distal (upper) end that may be used to coagulate the endocervix.

In order to prevent bowel herniation through the cervix, the cervix should be sutured closed at the completion of the procedure. This may be performed laparoscopically or vaginally, or using a combination of approaches. For example, a closure device, which is a long instrument with a sharp end on one side and a deployment mechanism (handle) on the other end, can be used for this purpose. The sharp end may open up (such as having two separate jaws) and be able to grasp suture material. The sharp end may also have a spring-loaded hook or notch that is exposed by engaging a mechanism on the handle. Closure of the cervix may be attained by loading a suture on the closure device, inserting the closure device up through the vagina and through the cervix. Once the needle is placed intra-abdominally (as visualized through the laparoscope), the suture is disengaged and the needle is withdrawn. The empty needle is then reinserted in a similar fashion through the contralateral side of the cervix, and the suture is loaded onto the needle, with the assistance of a laparoscopic grasping instrument. The closure needle device is then withdrawn into the vagina. The two ends of the suture and then tied down, which closes off the cervix.

In another embodiment, a blunt cylindrical device may be placed through the cervix. The diameter of the device would be approximately the same size of the morcellator device, which would therefore assist in maintaining the pneumoperitoneum. The blunt device may have a wider collar on the shaft of the device, which would prevent the device from advancing further into the abdominal cavity. Just distal to the collar, the device would have two holes, on opposite sides. A needle device could be deployed through these holes, which would advance through the cervical tissue and into the abdominal cavity. This needle device could be preloaded with a suture, or alternatively, a suture could be placed laparoscopically and loaded onto the needle device. If preloaded, one end of the suture would be left intra-abdominally. If loaded laparoscopically, one end of the suture would be withdrawn into the instrument and out through the vagina. After retrieving the first needle, the needle is advanced on the opposite side and the suture is grasped intra-abdominally, and brought out into the vagina on the opposite side. The suture would then be tied, closing off the cervical defect. Sutures could be placed laterally in the cervix, and then held without being tied. A second suture could then be placed anteriorly and posteriorly, and then both sutures tied down.

In another embodiment, the needle closure device advanced through the blunt device could be hollow and have a thin loop that is advanced through the end of the needle, once the needle is placed intra-abdominally. The needle could be advanced through one side of the upper portion of the probe, at an angle so that it engages the upper cervical tissue before it emerges into the peritoneal cavity. A suture placed laparoscopically, could then be placed through this “snare,” and the loop snare is brought back into the hollow needle, which would trap the suture in the needle closure device. The other end of the suture could be placed in a similar manner and the blunt device withdrawn, leaving the two suture ends in the vagina, which could then be tied down. Rather than a separate blunt probe, this or any of the cervical closure devices could be built in as part of the cervical trocar device, to minimize instrument exchanges.

In another embodiment, on one side a snare is placed through a needle, and on the other end, a hollow needle is inserted with a suture preloaded in the needle. The suture is then advanced through the needle, and a laparoscopic grasper is used to place the suture through the snare, which is then withdrawn, bringing both ends out through the vagina.

In another embodiment, the collar is located over the blunt central obturator placed in the cervix, which is placed up against the outer portion of the cervix. In the collar are one or more channels that may be parallel or near parallel to the shaft of the obturator. A needle may be placed through the channel in order to close the cervical defect. The needle may be notched or may have jaws that open up once the end has been placed into the peritoneal cavity. The suture may be pre-loaded onto the needle before placement in the channel, or the suture may be loaded onto the needle with laparoscopic assistance.

In another embodiment, a self-contained suturing device may be used to close the cervical defect. This concept is based on the CAPIO® suturing device (Boston Scientific Corp.) with some significant differences. The cored cervix may be closed by suturing it with a surgical suture applied by using a finger-guided suture device having a rotatably-driven, substantially semi-circular surgical needle designed for collecting the surgical suture via a distal portion of the surgical needle upon contact therewith and for retaining and guiding the surgical suture while suturing. The suture has small needle tips that are attached to both ends of the suture. The needle points are preloaded onto the end of the instrument, where they sit on the distal aspect of the curved needle shafts. There are two shafts that are located on either side of the instrument, so that they direct their needles in an opposite direction from each other. Alternatively, there may be only one needle shaft, and that would need to be loaded twice, once after the first needle is placed. The central loop of suture is contained within the shaft of the instrument so that it can not become entangled in the repair. The device is placed through the cervical defect either through a trocar device (as described previously) or by itself after the trocar has been removed. If placed through a trocar, the trocar is withdrawn partly into the cervical canal to expose the cervical tissue to be closed. The device is placed up through the cervix and when in position, the trigger is deployed, which throws one or both needle shafts (and therefore the needles attached to the sutures) through the tissue and the needle tip is then captured in the cage of the shaft of the instrument. The needle shaft then is retracted back into the instrument. The second needle pass is then made (unless done all at once with the first deployment of the trigger) which completes the suture passes. The instrument is then withdrawn out through the cervix and vagina and the needle tips are retrieved from the instrument cages. The needle tips may be cut off and the suture may be tied down, closing off the cervical defect.

The device described above may also be constructed so that it locks into the trocar in such a way that it exposes enough of the instrument beyond the trocar to allow the needle shaft to operate with the cage, which catches the needle tips. Once locked into place, the trocar is pulled back into the cervix until the needle will be driven into cervical tissue.

An absorbable plug with channels that run through the plug may be used to assist with hemostasis in the cervical channel. Once the cervical defect has been closed, the sutures are kept long and threaded through the plug and the plug is then tied down, which keeps the plug in the canal.

In another embodiment, an absorbable or nonabsorbable plug may be placed vaginally into the cervical defect with or without being sutured in place. This plug may be a slightly larger diameter than the defect, in order to create a tight fit, and the outer diameter of the plug may have ridges or other devices to keep it in place. The plug may have expandable wings that flare out like a Molly bolt on the inside of the cervix so that it does not fall out through the cervix. The plug may be or include a hydrogel.

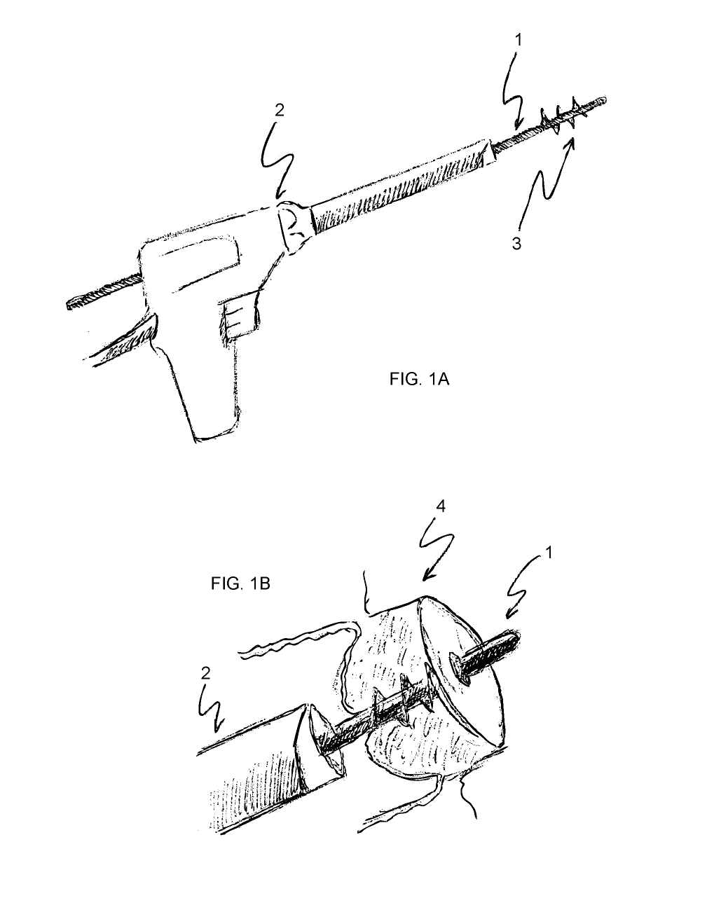

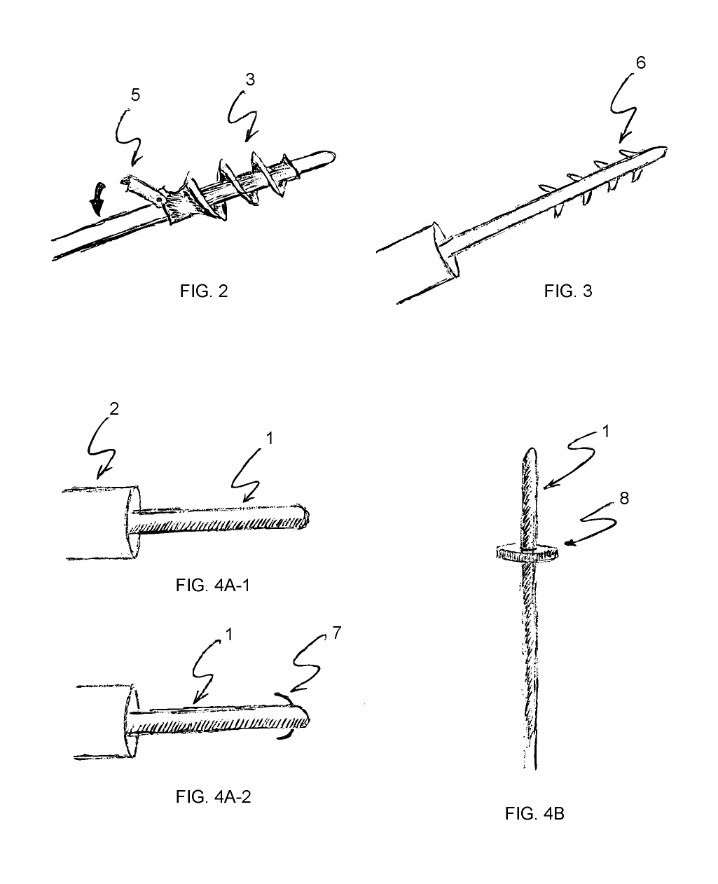

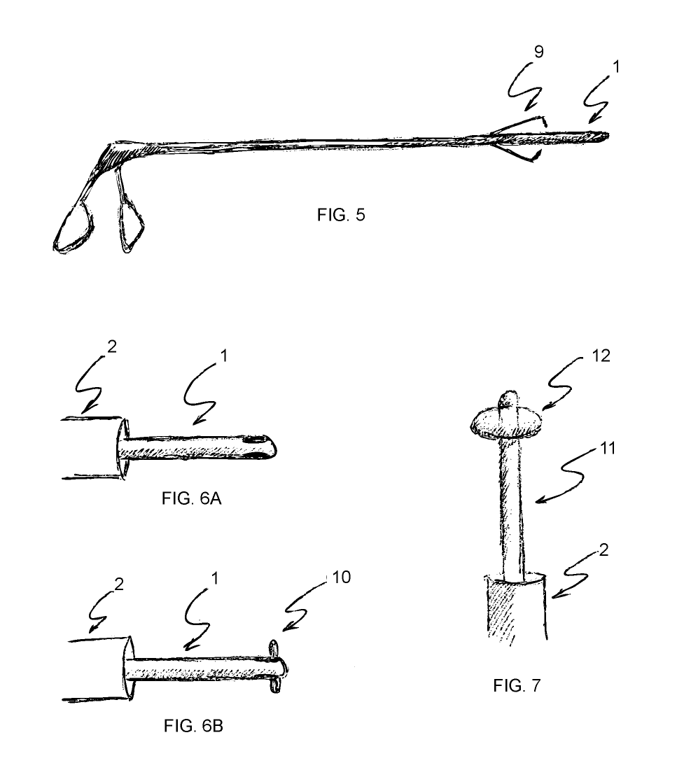

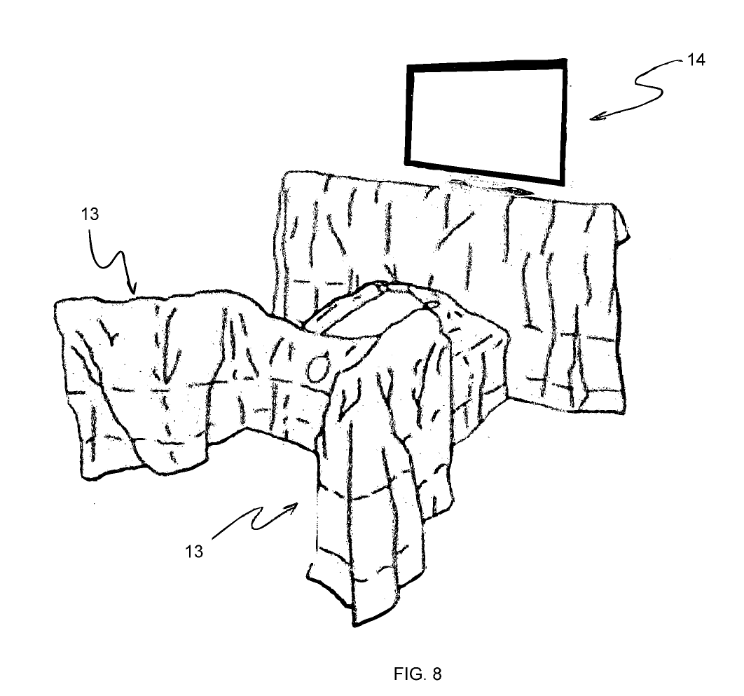

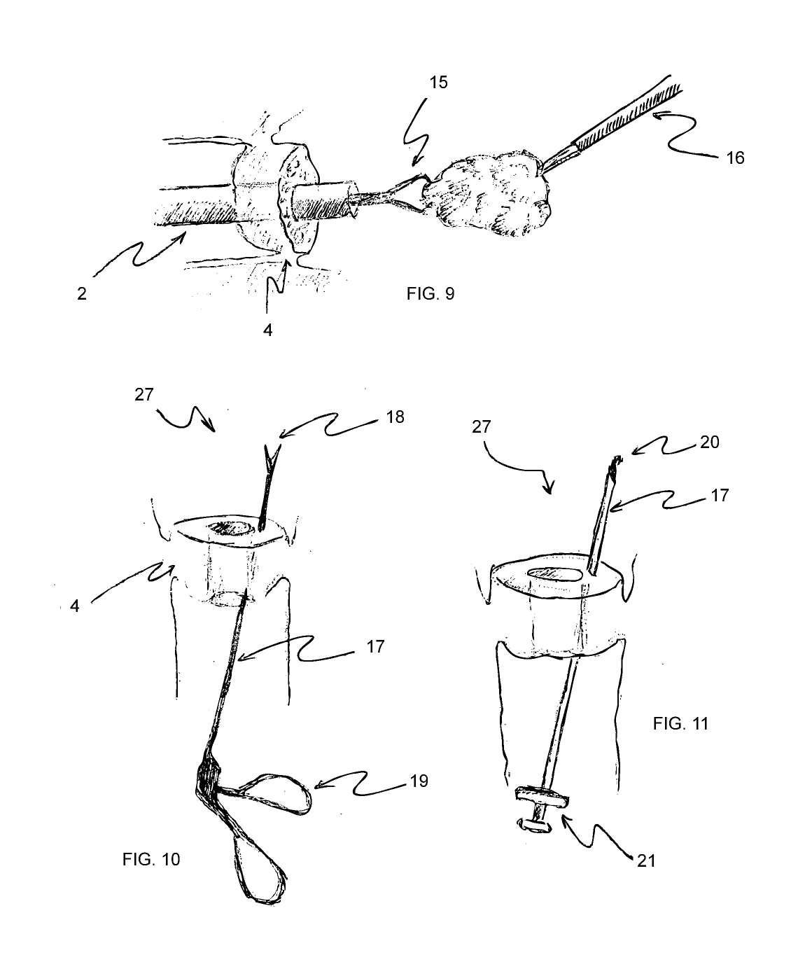

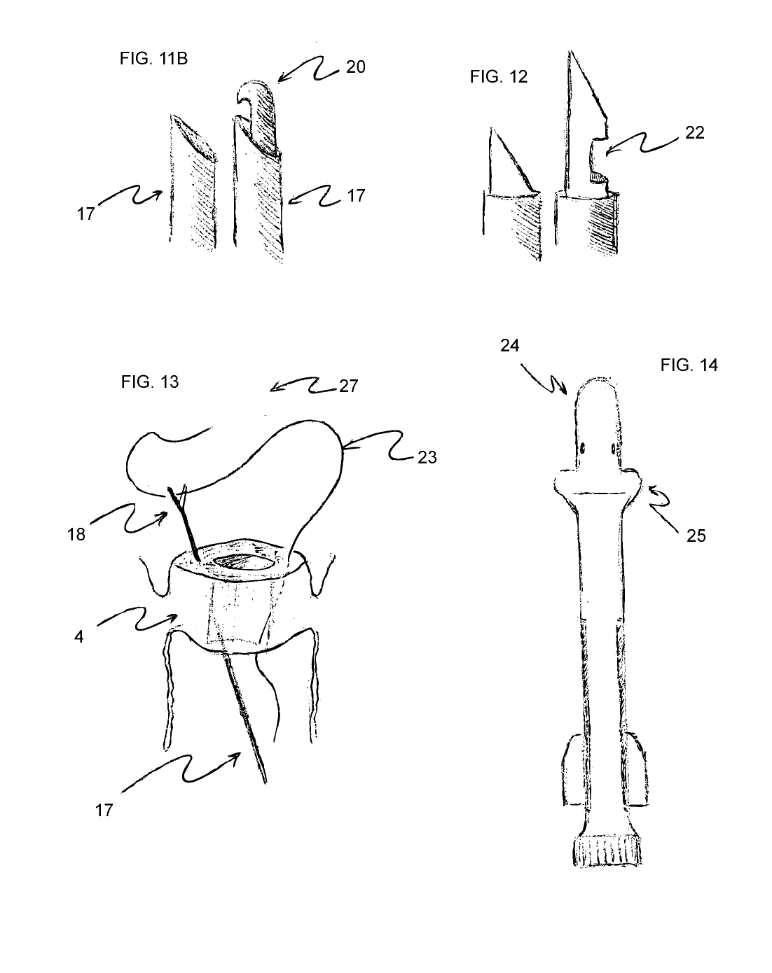

FIG. 1 illustrates the general design of the central rod instrument (1), which is placed inside the morcellator device (2). In this embodiment, the central rod has a flat screw (3) located in a fixed position near the distal end of the rod. The diameter of the widest portion of the screw must be smaller than the diameter of the morcellator blades. A stabilization ring may be placed on the rod, to prevent the morcellator from deviating along a straight pathway through the cervix. FIG. 1A demonstrates the rod placed through the morcellator and FIG. 1B shows a close-up view of the device placed inside the cervix (4).

FIG. 2 depicts a screw (3) that is adjustable along the length of the rod and may then be locked into position on the rod. In this example, flipping down a locking mechanism (5) fixes the screw into position, which may be changed by removing the rod and unlocking and relocking the screw in position.

FIG. 3 illustrates the use of barbs (6) located along the outer portion of the rod. These barbs allow the rod to be placed into the cervix, but when traction is applied to the cervix, the barbs engage and hold onto the cervical tissue.

FIG. 4A-1-4A-2 demonstrate the use of hooks (7) that are deployed at the distal end of the rod, after the rod has been placed through the cervix. These hooks grab the cervical tissue and assist in retrieval of the cervix after morcellation. FIG. 4B demonstrates a rod with a stabilization ring (8) along the shaft.

FIG. 5 demonstrates a device that has both a central rod (1) for placement through the cervix, and a grasping instrument (9) for holding on to the ectocervix. The grasping instrument may be a single-tooth tenaculum or other instrument that will allow a good grasping of the cervix, but also permits the jaws of the instrument to close enough so that the outer portion of the instrument does not interfere with the morcellation process.

FIGS. 6A-B illustrate a rod in which prongs (10) may be deployed at the distal end to engage the cervical tissue to facilitate removal of the cervix after coring out with the morcellator.

FIG. 7 illustrates a catheter (11) placed through the cervix which has a balloon (12) on the tip that can be inflated and pulled down against the cervix to prevent loss of pneumoperitoneum through the cored-out cervix.

FIG. 8 illustrates the room set-up where one surgeon is located between the patient's legs (13) and controls the morcellator device. The surgeon may look at a monitor (14) located at the head of the bed with a reversed image, which will allow the surgeon to see a “mirror-image” of the instrument, making the morcellation process easier.

FIG. 9 demonstrates the morcellation process, with the use of a grasping instrument (15) placed through the morcellator (2) which has been placed through the cored-out cervix (4) and the surgeon's assistant helping to stabilize the specimen with a laparoscopic grasping instrument (16).

FIG. 10 demonstrates a needle device (17) with jaws (18) that can be opened with the handle (19) on the other end, and is placed through the cervix (4) and up into the abdominal cavity (27), as visualized by the laparoscopic surgeon.

FIGS. 11 and 11B demonstrate a needle (17) with a spring loaded hook (20). After placement of the needle through the cervix and into the abdominal cavity (27), the hook is deployed by pressing on a spring loaded button on the opposite end (21), and suture may be loaded onto this hook.

FIG. 12 demonstrates a needle with a spring loaded notch (22). After placement of the needle through the cervix and into the abdominal cavity (27), the notch is exposed by pressing on a spring loaded button on the opposite end. This exposes the notch by withdrawing the outer sheath and the suture may be loaded onto this notch. Releasing the button traps the suture between the notch and the outer sheath.

FIG. 13 demonstrates the process of leaving one end of the suture (23) intra-abdominally (27) on one side of the cervix, and then placing a grasping needle (17) up on the other side of the cervix, and then withdrawing that end, which leaves both ends in the vagina, which can be tied down to close the cervical defect.

FIG. 14 shows a blunt tipped device (24) placed into the cervix with a larger collar (25) that pushes up against the outer portion of the cervix. A grasping or hollow needle (26) can be placed through one side of the instrument to deliver or retrieve suture material from the abdominal cavity (27) and through the cervical tissue.

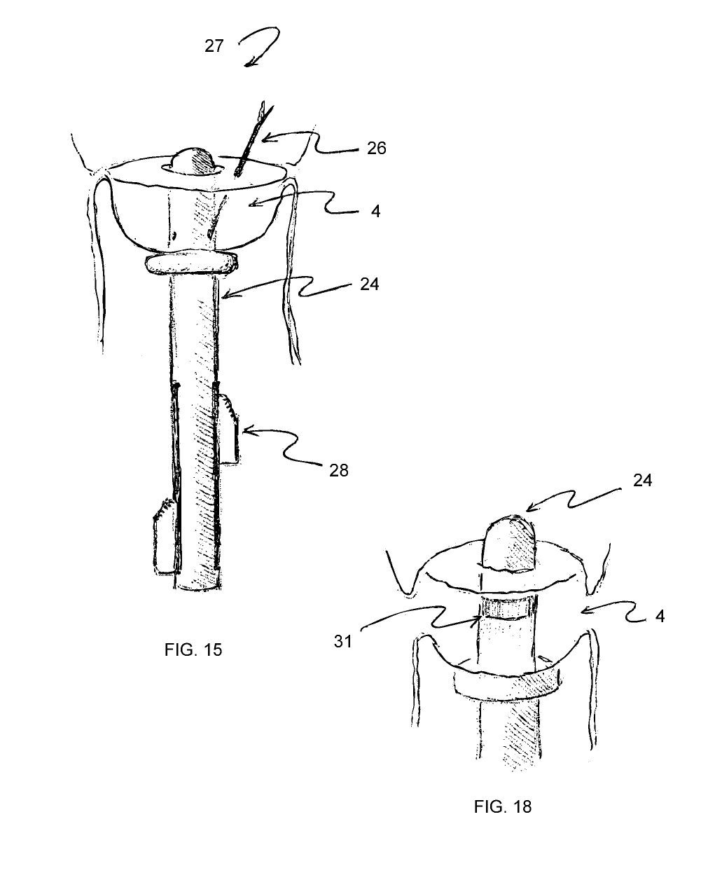

FIG. 15 shows a grasping needle (26) placed through the blunt probe (24) and through one side of the cervix (4) and into the abdominal cavity (27). This needle may be part of the instrument and is deployed by pressing a sliding mechanism (28) that pushes the needle through the tissue. This grasping needle is used to deliver or retrieve suture.

FIG. 16 demonstrates a hollow needle (29) emerging through the side of the blunt probe (24), and then another mechanism deploys a snare (30) through the needle in order to capture suture material (23). This process can be repeated on the other side. Both ends of the suture are then brought out through the cervix and into the vagina, and can be tied down to close the cervical defect.

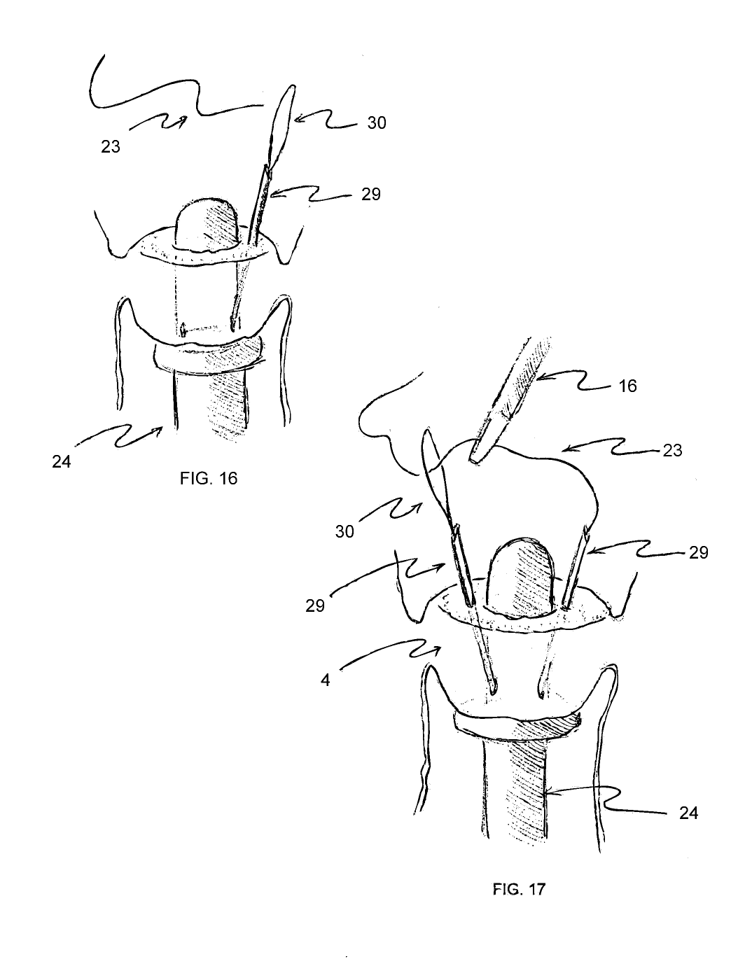

FIG. 17 illustrates a snare loop (30) on one side and a hollow needle (29) with a pre-loaded suture (23) within the needle on the other side of the blunt probe. The suture coming out from the hollow needle is then directed with the help of a laparoscopic instrument (16) through the snare on the opposite side. This end of the suture is then brought out through the cervix (4), and both ends are tied down, closing off the cervix.

FIG. 18 illustrates the electrocautery function of the blunt probe. This may involve a monopolar metallic ring (31) or two concentric rings that would employ bipolar energy, in order to coagulate the inner portion of the cervical core, if needed for control of bleeding. The probe may be advanced or withdrawn through the cervix to coagulate this tissue. Following this maneuver, closure of the cervix may be performed as described above.

FIG. 19 illustrates a separate handheld coring device (32) used to core out the cervical canal and surrounding tissue. This device is a hollow instrument with a sharp or serrated blade (33).

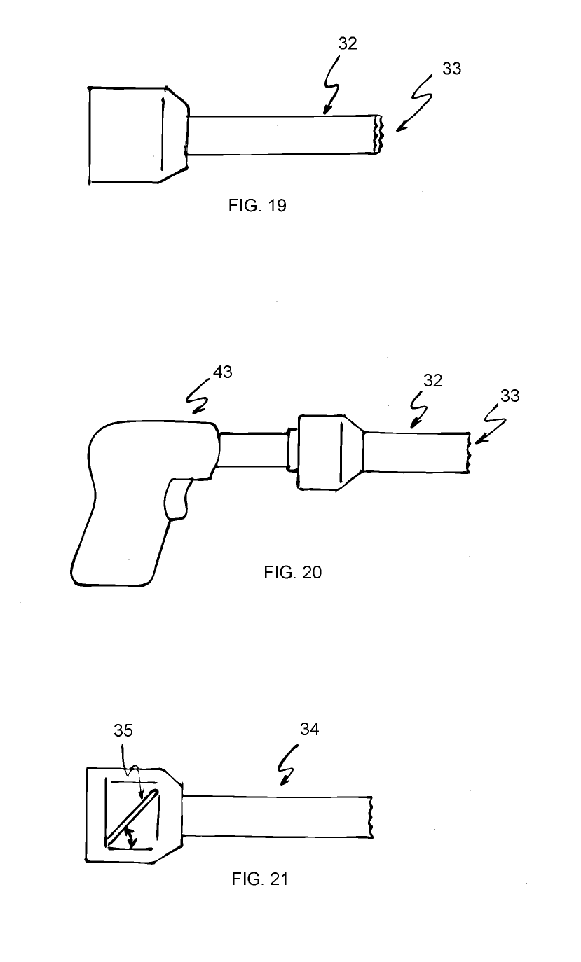

FIG. 20 illustrates a cervical coring hollow tube that is detachable from an electromechanical device (43). Once the hollow tube has cored out the cervix, it is detached from the electromechanical instrument. It may be unscrewed, or another type of attachment mechanism may be released, which separates the two instruments.

FIG. 21 illustrates a cervical trocar device (34) with a proximal valve (35) that is used to maintain a pneumoperitoneum when instruments are placed in or removed form the trocar.

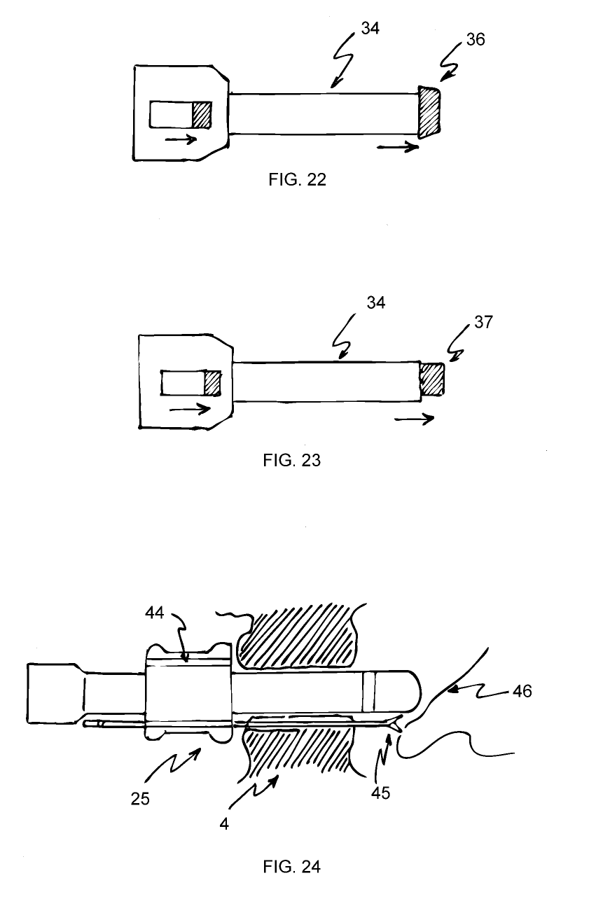

FIG. 22 illustrates a cervical trocar device (34) with a protective cover (36) applied over the sharp blade after coring out the cervix. This protective cover may be placed up inside the trocar and once it exits out the distal end of the trocar, it deploys out over the sharp blade.

FIG. 23 demonstrates a cervical trocar device (34) with a protective inner sleeve (37) inserted beyond the sharp blade.

FIG. 24 shows an obturator instrument in the cervix with a collar (25) that is brought up against the cervix (4). The collar has two channels (44) located on either side, through which a needle (45) can be placed and will then traverse the cervical tissue. The distal tip of the needle will then come through the cervix and can be used to deliver or retrieve suture (46) that is used to close the cervix.

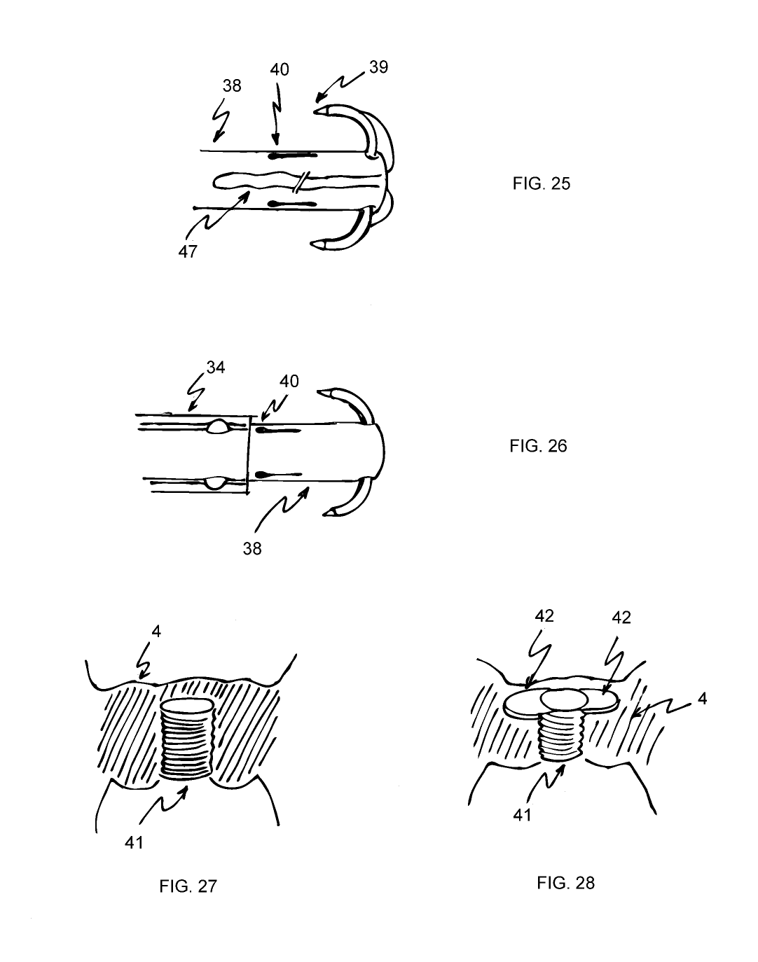

FIG. 25 demonstrates a suturing device (38) that automatically delivers a needle tip (39) through the cervical tissue and catches the needle tip in the cage (40), located in the shaft of the instrument. This can be done on either side of the cervical defect. The suture (47) is then tied down.

FIG. 26 demonstrates the attachment of the suturing device (38) to the trocar (34), which exposes the needle path and the cage (40) where the needle will be captured.

FIG. 27 illustrates a barrel-shaped plug (41) that can be placed in the cervical defect.

FIG. 28 illustrates a barrel-shaped plug (41) that has two wings (42) which flare out after being placed through the cervix.

### Example

A surgical procedure was performed according to the present disclosure as follows:

A 5 mm laparoscope was placed through the umbilicus, and 5 mm trocars were placed in the right and left lower quadrants.

A standard approach to LSH was performed, in this case using the Gyrus PK cutting forceps. The round ligaments were coagulated and a bladder flap was created. The utero-ovarian ligaments and tubes were then transected and the broad ligament was skeletonized, exposing the uterine vessels. The posterior leaf of the broad ligament was incised towards the uterosacral ligament, which further isolated the uterine vessels and allowed the ureters to fall further from the operative area. The uterine vessels were then coagulated, but not transected until the uterus became cyanotic, indicating successful bilateral occlusion of the vessels. A Gyrus PLASMASPATULA® cutting device was then used to transect the uterus from the cervix, and bleeding on the cervical bed was coagulated with bipolar cautery. The uterine manipulator was removed during the transaction of the uterus.

At this point, one of the surgeons left the side of the patient and repositioned between the patient's legs. A tenaculum was placed on the cervix and the cervix was sounded, in order to determine the proper axis of the endocervical canal. A 10 mm flat myoma screw was placed through a Gynecare MORCELLEX® morcellator, advanced transvaginally, and placed up against the cervical os. The instrument was slowly screwed into the cervix until it was visualized (laparoscopically) coming through the upper portion of the cervix. The morcellator was then applied to the cervix and, with gentle traction on the myoma screw, morcellation of the endocervical canal was performed until the core could be removed through the morcellator shaft. The morcellator was left in place and advanced until it entered the pelvic cavity.

Morcellation was then performed in standard fashion using, in this case, a 10 mm tenaculum, with the assistant feeding the specimen to the surgeon. A monitor placed at the head of the operating table, showing a mirror-image view, can greatly assist the surgeon performing morcellation.

Following completion of the morcellation, the canal was closed to prevent herniation of bowel through the tunnel created by the morcellator. This was accomplished with a fascial closure needle and absorbable suture, in this case, 0-PDS suture. (A variety of other closure techniques are disclosed herein.)

We claim:

1. A hysterectomy method comprising the following steps in the recited order:
severing a patient's uterus from the patient's cervix; then
coring the cervix by advancing a transcervical trocar through the cervix, wherein the transcervical trocar comprises a distal cutting edge, thereby coring that portion of the cervix which is encompassed by the distal cutting edge; and then
morcellating the severed uterus by leaving the transcervical trocar in the cored cervix, extending a grasping device through the trocar to grasp the severed uterus, and pulling the severed uterus into the trocar while rotating the trocar.
